(19) United States
(12) Patent Application Publication
(10) Pub. No.: US 2024/0358436 A1
(43) Pub. Date: Oct. 31, 2024

(54) AUGMENTED REALITY SYSTEM AND METHOD WITH PERIPROCEDURAL DATA ANALYTICS

(71) Applicant: MEDIVIEW XR, INC., Cleveland, OH (US)

(72) Inventors: Jeffrey H. Yanof, Solon, OH (US); Aydan Thomas Hanlon, Akron, OH (US); Ross Daniel Hinrichsen, Minneapolis, MN (US); Peter Nicholas Braido, Wyoming, MN (US); Gabrielle Stefy, Voorhees, NJ (US); Michael Russel Evans, Conway, AR (US); Charles Martin, III, Pepper Pike, OH (US)

(21) Appl. No.: 18/645,629

(22) Filed: Apr. 25, 2024

Related U.S. Application Data
(60) Provisional application No. 63/498,039, filed on Apr. 25, 2023.

(51) Int. Cl. A61B 34/10; A61B 34/00; A61B 34/20; A61B 90/00; A61B 90/50; G03H 1/00; G06V 10/70; G06V 20/20

(52) U.S. Cl. CPC A61B 34/10; A61B 34/20; A61B 34/25; A61B 90/36; A61B 90/50; G03H 1/0005; G06V 10/70; G06V 20/20; A61B 2034/105; A61B 2034/2065; A61B 2090/365; A61B 2090/502; G06V 2201/03

(57) ABSTRACT

The present disclosure includes methods for improving surgical precision and outcomes in medical procedures through the use of an augmented reality system. The method involves generating a preoperative plan by visualizing a holographic representation of a patient's anatomy within an augmented reality environment, recording procedure data based on the preoperative plan, updating the augmented reality system using the recorded data, and applying the updated system to establish a continuous improvement feedback loop. This approach enhances surgical accuracy and patient outcomes by integrating real-time data feedback into the surgical process, thereby optimizing surgical procedures and promoting continuous improvement in medical practices.

### CROSS-REFERENCE TO RELATED APPLICATIONS

This application claims the benefit of U.S. Provisional Application No. 63/498,039, filed on Apr. 25, 2023. The entire disclosure of the above application is hereby incorporated herein by reference.

### FIELD

The present disclosure relates to holographic augmented reality applications and, more particularly, medical applications employing holographic augmented reality.

### INTRODUCTION

This section provides background information related to the present disclosure which is not necessarily prior art.

Image-guided surgery has become an invaluable tool across a diverse range of medical interventions. By visually correlating intraoperative data with preoperative data, image-guided surgery provides an opportunity to enhance the surgeon's ability to navigate complex procedures with greater accuracy. The integration of this technology into surgical practices has been instrumental in elevating both the safety and the efficacy of numerous surgical procedures, thereby improving patient outcomes.

While image-guided surgery promises to be transformative in the field of medical interventions, its implementation is not without its challenges and limitations. One significant issue is the manner in which preoperative and intraoperative data are presented to the practitioner. This data, crucial for the guidance of surgical instruments, is typically displayed on two-dimensional (2D) monitors that surround the patient's operating table. Such a setup can inadvertently cause the practitioner's attention to be diverted away from the patient and towards the static displays. This division of focus can be detrimental, as it may affect the practitioner's ability to perform the surgery with the highest level of precision and care. The reliance on 2D displays to convey complex spatial information about the patient's anatomy can also lead to misinterpretation of the data, potentially impacting the surgical performance and outcomes.

Furthermore, the physical setup of traditional image-guided systems can pose ergonomic challenges that may affect the practitioner's comfort and performance during surgery. For instance, the necessity for the practitioner to repeatedly shift their gaze from the patient to the 2D displays and back can lead to physical strain. Specifically, the practitioner's neck and back are subjected to stress due to the constant need to adjust their line of sight between the surgical site and the monitors. Over time, this can not only cause discomfort and potential long-term musculoskeletal issues for the practitioner but can also momentarily distract them during critical phases of the surgery, which may compromise the procedure's success and safety.

There is a continuing need for a visualization, guidance, and navigation method and system for a procedure that involves holographic augmented, mixed, and/or extended reality. Desirably, the method and system also allows the practitioner to generate, view, and interact with operating data and the patient within the same field of view.

### SUMMARY

In concordance with the instant disclosure, a visualization, guidance, and navigation method and system for a procedure that involves holographic augmented, mixed, and/or extended reality, and which allows the practitioner to generate, view, and interact with operating data and the patient within the same field of view, have been surprisingly discovered.

In one embodiment, the present disclosure relates to a method for performing a procedure. For example, a practitioner can perform and update a medical procedure on a patient by utilizing an augmented reality system. Initially, the practitioner can provide a procedural plan that outlines the steps and strategies for the medical procedure. During the actual performance of the medical procedure, the practitioner can use the augmented reality system, which is designed to assist by adhering to the procedural plan. As the procedure progresses, the augmented reality system can record specific metrics that capture various aspects of the surgical process. These metrics can be crucial for understanding the procedure's dynamics and for identifying areas that may benefit from improvements. Once the procedure is completed, the practitioner can use the augmented reality system to update the original procedural plan. This update is based on the metrics that were recorded during the procedure, allowing for the refinement of the plan to better suit the realities of the surgical environment and patient-specific factors.

In another embodiment, the method involves performing and updating a medical procedure on a patient using an augmented reality system that provides a practitioner with intraoperative guidance. This method includes providing a procedural plan, utilizing the augmented reality system to perform the medical procedure, and alerting the practitioner to any deviations from the procedural plan through visual or auditory cues. The system includes a head-mounted display that renders a holographic representation of the medical procedure, overlaying it within the practitioner's view of the patient. Metrics are recorded during the procedure, which include data such as instrument positioning and operative action metrics. These metrics are used to update the procedural plan, thereby establishing a feedback loop that refines the procedural steps for future medical procedures. The method also involves analyzing postoperative results to compare outcomes and ascertain the effectiveness of the instrument placement, with the augmented reality system further configured to provide a postoperative review interface for displaying recorded metrics and adjusting the procedural plan.

In a further embodiment, the augmented reality system is designed to enhance the precision and outcomes of medical procedures by utilizing a head-mounted device equipped with a display capable of rendering holographic images. This device incorporates a processor and non-transitory memory, which work in conjunction with a tracking system to provide real-time data on the position and orientation of a surgical instrument relative to a patient's anatomy. The system's machine-readable instructions facilitate the transformation of tracking and pre-operative image data into a unified coordinate system, enabling the generation of a three-dimensional holographic representation of both the patient's anatomy and the surgical instrument. This holographic overlay, presented within the practitioner's field of view, serves as an intraoperative guide. Additionally, the system records intraoperative metrics to update procedural plans, thus establishing a feedback loop that contributes to the continuous improvement of surgical practices. The system also includes a communication interface for data exchange and a user interface that allows practitioners to adjust procedural plans and engage with the holographic representations during medical procedures.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1 is a flowchart of a method for performing a medical procedure using an augmented reality system, according to one embodiment of the present disclosure.

FIG. 2 is a flowchart of a method for performing a medical procedure using an augmented reality system, according to one embodiment of the present disclosure.

FIG. 3 is a block diagram illustrating a holographic augmented reality visualization and guidance system, according to one embodiment of the present disclosure.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature unless otherwise disclosed, and thus, the order of the steps can be different in various embodiments, including where certain steps can be simultaneously performed.

Unless otherwise defined, all technical terms used herein have the same meaning as commonly understood by one of ordinary skill in the art to which the present disclosure pertains.

As used herein, the terms “a” and “an” indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a process reciting elements A, B and C specifically envisions embodiments consisting of, and consisting essentially of, A, B and C, excluding an element D that may be recited in the art, even though element D is not explicitly described as being excluded herein.

As referred to herein, disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping, or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, the terms “interventional device” or “tracked instrument” refers to a medical instrument used during the medical procedure.

As used herein, the term “tracking system” refers to something used to observe one or more objects undergoing motion and supply a timely ordered sequence of tracking data (e.g., location data, orientation data, or the like) in a tracking coordinate system for further processing. As an example, the tracking system can be an electromagnetic tracking system that can observe an interventional device equipped with a sensor-coil as the interventional device moves through a patient's body.

As used herein, the term “tracking data” refers to information recorded by the tracking system related to an observation of one or more objects undergoing motion.

As used herein, the terms “imaging system,” “image acquisition apparatus,” “image acquisition system” or the like refer to technology that creates a visual representation of the interior of a patient's body. For example, the imaging system can be a computed tomography (CT) system, a fluoroscopy system, positron emission computed tomography, magnetic resonance imaging (MRI) system, an ultrasound (US) system including contrast agents and color flow doppler, or the like.

As used herein, the terms “coordinate system” or “augmented realty system coordinate system” refer to a 3D Cartesian coordinate system that uses one or more numbers to determine the position of points or other geometric elements unique to the particular augmented reality system or image acquisition system to which it pertains. For example, 3D points in the headset coordinate system can be translated, rotated, scaled, or the like, from a standard 3D Cartesian coordinate system.

As used herein, the terms “image data” or “image dataset” or “imaging data” refers to information recorded in 3D by the imaging system related to an observation of the interior of the patient's body. For example, the “image data” or “image dataset” can include processed two-dimensional or three-dimensional images or models such as tomographic images, e.g., represented by data formatted according to the Digital Imaging and Communications in Medicine (DICOM) standard or other relevant imaging standards.

As used herein, the terms “imaging coordinate system” or “image acquisition system coordinate system” refers to a 3D Cartesian coordinate system that uses one or more numbers to determine the position of points or other geometric elements unique to the particular imaging system. For example, 3D points and vectors in the imaging coordinate system can be translated, rotated, scaled, or the like, to the Augmented Reality system (head mounted displays) 3D Cartesian coordinate system.

As used herein, the terms “hologram”, “holographic,” “holographic projection”, or “holographic representation” refer to a computer-generated image stereoscopically projected through the lenses of a headset. Generally, a hologram can be generated synthetically (in an augmented reality (AR)) and is not a physical entity.

As used herein, the term “physical” refers to something real. Something that is physical is not holographic (or not computer-generated).

As used herein, the term “two-dimensional” or “2D” refers to something represented in two physical dimensions.

As used herein, the term “three-dimensional” or “3D” refers to something represented in three physical dimensions. An element that is “4D” (e.g., 3D plus a time and/or motion dimension) would be encompassed by the definition of three-dimensional or 3D.

As used herein, the term “integrated” can refer to two things being linked or coordinated. For example, a coil-sensor can be integrated with an interventional device.

As used herein, the term “real-time” or “near-real time” refers to the actual time during which a process or event occurs. In other words, a real-time event is done live (within milliseconds so that results are available immediately as feedback). For example, a real-time event can be represented within 100 milliseconds of the event occurring.

As used herein, the terms “subject” and “patient” can be used interchangeably and refer to any vertebrate organism.

As used herein, the term spatial “registration” refers to steps of transforming virtual representation of tracked devices-including holographic guides, applicators, and ultrasound image stream-and additional body image data for mutual alignment and correspondence of said virtual devices and image data in the head mounted displays coordinate system resulting in a stereoscopic holographic projection display of images and information relative to a body of a physical patient during a procedure, for example, as further described in U.S. Patent Application Publication No. 2018/0303563 to West et al., and also applicant's co-owned U.S. patent application Ser. No. 17/110,991 to Black et al. and U.S. patent application Ser. No. 17/117,841 to Martin III et al., the entire disclosures of which are incorporated herein by reference.

The present technology relates to ways for providing holographic augmented reality visualization and guidance in performing a medical procedure on an anatomical site of a patient by a user. Systems and uses thereof can include an augmented reality system, a tracked instrument, an image acquisition system, and a computer system. The present disclosure is also directed to a method of utilizing the system and the feedback loop to plan and analyze medical procedures.

In one embodiment, the procedure can begin with preoperative planning. This can include where the practitioner uses the AR system to visualize a holographic representation of the patient's anatomy. This representation is generated from preoperative imaging data, such as CT or MRI scans, and is accurately overlaid onto the patient's body in the AR environment. The practitioner can interact with this holographic model to plan the surgical approach, identify potential challenges, and determine the optimal entry points and paths for surgical instruments. In other embodiments, the method can start with a procedure performed by a practitioner without preoperative planning. In other embodiments, the procedural plan can be based on knowledge of the practitioner.

During the procedure, the AR system can provide real-time guidance by displaying the planned approach and critical anatomical structures within the field of view of the practitioner. The system can track the position and orientation of surgical instruments, which allows the practitioner to confirm that the instrument is aligned with the preoperative plan. If the instruments deviate from the intended path, the system can alert the practitioner with visual or auditory cues, allowing for immediate correction. Additionally, the system can be recording various metrics, as described herein, during the procedure.

After the procedure, the system can facilitate a postoperative review where the practitioner can assess the surgery's success and compare the actual outcomes with the preoperative plan. This review can include analyzing the precision of instrument placement, the effectiveness of the intervention, and the overall efficiency of the procedure. The review can also include analyzing the recorded metrics relative to the preoperative plan. The effectiveness of the procedure can be objectively determined. For example, additional imaging after the procedure can be used to determine if the surgical intervention, such as ablation, was successful.

The postoperative review can include an affirmative selection to the updates made to the procedural plans. For example, the practitioner can made in situ changes to the procedural plan such as edits to one particular step or the addition of a new step that was not considered during the preoperative planning process. During the postoperative review process, the practitioner can review the changes made to the procedural plan to determine whether the procedural plan should be updated. The post operative analysis can allow the practitioner to perform customized procedures without affecting the standing procedural plan. The system can allow for the overlay of postoperative imaging data, such as CT or MRI scans, to evaluate the effectiveness of the intervention against the preoperative holographic models. This comparison helps in determining the success of tumor resections, ablations, or other targeted treatments by providing a clear visual representation of the treated area before and after the procedure.

The insights gained from the postoperative analysis can then be fed back into the system. This feedback loop can involve updating the system's algorithms to improve the accuracy of the holographic representations and the effectiveness of the intraoperative guidance. For example, if the analysis reveals that certain instrument paths led to better outcomes (e.g., more effectively treat the identified tumor), the system can incorporate this data to suggest improved paths for future surgeries.

When the next procedure is performed, the updated system can apply the refined algorithms and enhanced AR content to provide even more accurate guidance. The continuous feedback loop ensures that each procedure benefits from the accumulated knowledge and experience, leading to a cycle of ongoing improvement in surgical practices and patient care.

It should be further appreciated that, in certain embodiments, the system can be updated in real time. As the practitioner interacts with the patient and the system, the system can be constantly updating projections and predicted outcomes.

Over time, as more data is collected and analyzed, the system becomes increasingly sophisticated, learning from a wide array of procedures and outcomes. This long-term adaptation contributes to the development of best practices and can potentially lead to predictive analytics, where the system can anticipate challenges and suggest preemptive actions to the surgeon.

The holographic augmented reality visualization and guidance system can include multiple data streams. These data streams can include inputs, such as user data, imaging data, and tracking data from external sources. The data streams can also include various outputs, such as visuals to various external devices and data recording that can be used in subsequent applications. It should be appreciated that the data streams can interact in ways that can form one or more feedback loops in the holographic augmented reality visualization and guidance system, for example. Each feedback loop can include a process for collection, interpretation, and analysis of procedural data for building and improving data models.

In one non-limiting example, the holographic augmented reality visualization and guidance system and the associated feedback loop can be utilized in situ during a procedure. The feedback loop can include the collection of intraoperative data. The holographic augmented reality visualization and guidance system can then interpret and display the data on a common platform for further use. Guidance, navigation, and analytical data can be projected with the augmented reality system in proximity or registration with the physical patient. The sensorized tools can be shown relative to each other, via holographic projections of the holographic augmented reality visualization and guidance system, with their inclusion in the common area offering unique opportunities such as changing visuals or triggering sound effects when they are intersecting. The system can include haptics such as vibration and more advanced like force/pressure feedback.

In certain examples, the system can track a variety of metrics to enhance the surgical process and outcomes. The system can track instrument positioning metrics including X, Y, Z coordinates of the surgical instruments relative to the patient's anatomy; orientation and rotation angles of the instruments; path and trajectory followed by the instruments during the procedure.

The system can track instrument interaction metrics including frequency and duration of instrument intersections with the ultrasound field; and distance between an instrument tip and target tissues or structures. The system can track operative action metrics including needle insertion angle relative to the ultrasound probe and anatomical structures; number of needle insertions and repositions; speed and acceleration of the needle or other surgical instruments; and duration of specific surgical actions or phases of the procedure.

In particular examples, the system can track surgeon performance metrics including steadiness or shakiness of the practitioner hands during instrument manipulation; ergonomic metrics such as the practitioner posture and hand positioning; and practitioner reaction time to visual or auditory cues provided by the system.

The system can record visual attention metrics including eye-tracking data to determine where the surgeon's gaze is focused during the procedure; duration of gaze on specific visualizations or data points; and frequency of gaze shifts between the patient and the AR display.

In yet other examples, the system can record communication metrics including voice communication patterns among the surgical team; and commands issued, and responses received during the procedure. The system can record procedure efficiency metrics including overall procedure time from start to completion; time taken for setup and preparation; and periods of inactivity or downtime during the surgery.

The system can record outcome-related metrics including correlation between instrument usage patterns and surgical outcomes; postoperative recovery metrics such as healing time and complication rates; patient-specific outcomes linked to the surgical technique and instrument handling. The system can record environmental metrics including room temperature and lighting conditions during the procedure; and noise levels and potential distractions in the operating room.

Also, the system can record system interaction metrics including frequency and types of system alerts issued during the procedure; and surgeon's responses to system-generated recommendations or warnings. These metrics can be collected, analyzed, and used to inform the feedback loop, leading to continuous improvements in the system's performance and the surgical procedures it supports.

It should be appreciated that the holographic augmented reality visualization and guidance system can advantageously record both operative actions and metrics. Using the data sources and their interactions, different actions and metrics can be assessed. For example, the actions and metrics can include a needle approach angle, a number of times the needle intersected an ultrasound image, a speed of needle approach, relative movement of the operator's hands (e.g., a measure of how steady/shaky the operator's hands and tools were during use).

Other examples of operative actions and metrics include measurement and recording of parameters such as time, distance, and position relative to various aspects present and occurring during the surgical procedure, where such aspects can include one or more surgical or imaging tools, operator(s), portions of the patient anatomy, among other aspects found in a procedural room or surgical theater during a surgical procedure. The machine learning algorithm can determine the best or preferred settings for the surgical application under consideration such as: which anatomical holograms should initially be shown or hidden at each stage of the procedure; should the co-registered holograms be initially co-projected to physical patient or floating above the patient; which structures, e.g., vessels, tumor, organ, etc. should be used to adjust the registration between the ultrasound and CT holograms; and what are the highest risks in terms of critical structures to be avoided for a given surgical task.

The holographic augmented reality visualization and guidance system can further analyze comparable data and outcomes. This can include comparison to a predetermined surgical plan and/or data from one or more comparable procedures by the same or a different operator. Once enough data has been collected, it can be linked to overall metrics or positive/negative outcomes. For example, the system can assess whether multiple needle approaches significantly increased surgery time, as well as certain surgical instruments or actions trend towards either positive or negative outcomes. The outcome data can be accessed via a Fast Healthcare Interoperability Resources (FHIR®), a standard for exchanging healthcare information electronically that is set by Health Level Seven (HL7®) International, Inc. located in New Jersey USA, for example, and displayed with the system in proximity with the surgical field.

The holographic augmented reality visualization and guidance system can then update systems and behavior based on the recorded data. The insights can be used to update the system for alerts, warnings, and recommendations based on desired outcomes. For example, needle positions with a typically poor outcome can be signified as riskier based on parameter settings of a tumor ablation modality relative to the trajectory and/or multiple adjacent ablations.

For each procedure, the set of needle positions and their predicted ablation zones can be recorded relative to the imaged anatomical targets. In some cases, optimized trajectories can be predicted and displayed to the user as suggestions of how to proceed. The feedback loop can and should continue developing as more data is gathered. In further iterations, the new feedback provided to users can be assessed for usefulness and accuracy in addition to other new metrics.

In a further example, the holographic augmented reality visualization and guidance system can be utilized to track a surgeon or operator and their interactions with the system and/or with each other. The holographic augmented reality visualization and guidance system can allow for collection of unique metrics involving the surgeon's interactions and movement. These interactions can include eye tracking (including which visualizations were in focus), hand and finger tracking, head position and rotation tracking, and voice communication. For example, the system can determine that a practitioner that looks at live imaging more often can shorten procedure time.

The holographic augmented reality visualization and guidance system can record changes made to the preoperative plan by a practitioner during the procedure. For example, a practitioner can adjust an ablation zone based on their own experience. The system can record this change and utilize it in later iterations of the procedure, thereby, allowing the system to learn from the experience of the various practitioners that utilize the system.

Advantageously, these metrics can be utilized in a feedback loop process. Insights from analysis of data include addressing certain issues, including, as non-limiting examples: which types of data and visualizations are most helpful; where and what did the user's eye gaze dwell on what may have been avoided during the procedure; ergonomics such as preferred layout and orientation of visualizations to reduce operator strain and setup time; voice and spatial proximity of multiple operators within a session; periods of downtime/confusion and potential sources thereof; and hand interactions with tools (i.e., how often certain tools are used, what kind of approaches/grips are common, etc.).

The data streams of the holographic augmented reality visualization and guidance system can include pre-procedure analytics, intra-procedure analytics, and post-procedural analytics that can be used to inform the various further procedures performed with the holographic augmented reality visualization and guidance system. These analytics can be further understood by way of the examples presented herein.

It should be appreciated that the input streams of the system can include input from a plurality of holographic augmented reality visualization and guidance systems in communication with the common platform. The feedback loop procedures can be updated in real time as procedures at various locations are completed. The system can also register full body avatars for telecommunication and lifelike guidance. The system can allow for registration to anatomy in 3D and control of system from an external source. This registration can also apply to images, surgical tools, and the patient with a digital twin in the local space, and full set of twins in one or more remote location. All of the objects can be registered based on various imaging data, such a cone-beam computed tomography data, as the origin and displayed in VR or AR remotely.

During a procedure, data can be gathered and collated from various inputs by the holographic augmented reality visualization and guidance system. This can include interactions between tools, clinicians, and imaging, as non-limiting examples. This information can be used to record or report or display using augmented reality different aspects of the procedure for the user's review, for example: user profiles and behavior with needle/probe type, settings/parameters, trajectories, time between needle sticks, ablation time, total time, length of procedure, number of ablations, and the like; a replayable recording of tracked tools, UX elements, and physician interaction; 3D spatial visualization of data such as contours, percent surface area or volume coverage, heat map as defined by the location and time a tool or gaze spent in a particular area not to be confused with the actual heat of an ablation, playback, search like cases/results with similarity measure, eye tracking/shut off warnings/reduction of cognitive overload, trajectories of multiple probes/devices, and the like; and intentionally-recorded data such as saved pictures, videos, and voice memos that can be stored in a searchable database for recalling and comparing within a later procedure, and providing summaries in the AR display.

To enable holographic display, all data can pass through a central server of the holographic augmented reality visualization and guidance system and can be intentionally sent in a structured format. This can allow time series data describing the current state of the system and all connected endpoints. This data can be stored locally in a database, with options to export to common formats such as *.CSV and *.JSON files. The flexibility from this logging structure can allow for creation of reports and queries for a variety of outputs, as well as metrics between logs such as timestamps. For example, comparing timestamps of ablations could allow assessment of planning time for each placement. This can then be tied back to spatial location of the probe at each point to map difficulty of placement across the procedure. Secondary interactions between these inputs are only possible in a system that creates a common data relationship, such the present technology. For example, in a tumor ablation, the probe positions can be used to estimate a combined treatment zone and its coverage relative to the planned treatment zone including margins.

Statistical methods can be employed comparing learning curves, milestone time points, accuracy of therapy, etc. within and across users by means, ranges, standard deviations, conformity, t-tests and MANOVA (multivariate analysis of variance). Spatial 3D heat maps and pathways can be examined with Principal Component Analyses or types of regression (e.g., non-linear or linear regression for a curved versus a straight trajectory). Such methods can be particularly useful in applications with highly deformable tissue relating to a model derived from imaging, such as cardiac/cardiology, pulmonary/lung, and breast treatments, as further described herein.

In cardiology, the anatomy and physiology are such that not only is soft tissue involved, but the tissue is also constantly moving from respiration and heart rate. Additionally, the tissue actively interacts with dynamically changing environments in fluid flow, for example. It is for such reasons that the 3D/4D (time) visual representation of the hologram, spatial data, spatial sound, and metaverse avatars can be beneficial. In particular, the holographic augmented reality visualization and guidance system can be beneficial in use during a transcatheter aortic and mitral valve repair or replacement, pericardiocentesis, etc.

The holographic augmented reality visualization and guidance system can further utilize spatial analytics to assist with the dynamic nature of the cardiac anatomy. Three-dimensional or 3D data such as doppler blood flow through a valve (computation fluid dynamics and leaflet finite element analyses) can combine with time series data, combine with historical data, compare and contrast data streams. For example, one would be able to pick out a voxel and track its path through an aortic valve in a digital twin, into a coronary sinus, and around a vessel restriction (that afterwards would be able to do the same for a heart valve replacement and/or coronary stenting). Given enough data one could start to be predictive and prescriptive using Monte Carlo and/or Principal Component Analyses techniques (such as what is done for Statistical Shape Modeling of a patient population). Advantageously, this can all be performed from the perspective of a remote avatar and/or controlling a robotic system, as desired.

The holographic augmented reality visualization and guidance system can utilize spatial audio to assist with the dynamic nature of the cardiac anatomy, for example. 3D/4D spatial sound can be used to show where the sound is coming from graphically and can be represented by intensity level. Amplified and focal sound, such as drawing or circling around an area of indication can be hyper-audible. Another application is stereo audio as when an operator would be on the voxel or in the heart hearing 3D sound of the surrounding structures much like what is done in a theater with “surround” sound. The holographic augmented reality visualization and guidance system can allow an operator to visualize sound separation, such as frequencies of a conjoined twins child's heartbeat. The holographic augmented reality visualization and guidance system can allow an operator to visualize an acoustic decay of a mechanical heart valve such as indicating fracture initiation sites. The holographic augmented reality visualization and guidance system can provide haptic feedback based on spatial sound analysis of identified areas of interest.

### EXEMPLARY APPLICATIONS

Without being limited to any particular application, it should be appreciated that the system and feedback loop of the present disclosure can be utilized in various exemplary applications as described further herein.

### Application 1: Recording and Reporting of Spatial Time-Series Metrics

Pulmonary anatomy and physiology is such that not only is it soft tissue, but it is moving from respiration and heart rate. Additionally, the tissue actively interacts with dynamically changing environments in air flow, for example. It is for such reasons that the 3D/4D (time) visual representation of the hologram, spatial data, spatial sound, and metaverse avatars are beneficial. The holographic augmented reality visualization and guidance system can allow for AR tracking of flexible devices such as an EBUS (Endobronchial Ultrasound), a flexible catheter with fiber optic sensors, or biopsy catheter, and display of optical camera, depth camera, and/or ultrasound live. The holographic augmented reality visualization and guidance system can display live updates to deformation via photogrammetry, videogrammetry, ultrasound, LIDAR, and the like. The holographic augmented reality visualization and guidance system can provide heat maps of tissue deformation relative to target and the effect of the medical device. The holographic augmented reality visualization and guidance system can allow an operator to visualize individual lobe motion (Finite Element Analysis) relative to flow (Computation Fluid Dynamics) displayed in AR with data metrics. The holographic augmented reality visualization and guidance system can allow an operator to utilize multi-target or multi-probe integration.

The holographic augmented reality visualization and guidance system can allow the operator to predict a respiratory phase from a CT dataset (assuming a breath hold was used during the data acquisition). This can allow the operator to visualize intra-procedural respiratory phase matching. For example, a breath hold phase can be predicted by CT data set analysis. In certain examples, the system can utilize an electromagnetic or other motion sensor to track respiratory motion.

In breast treatment the tissue is highly malleable, and the tumors as well as adjacent structures change position over the respiratory cycle, posture, and interaction of tools. Additionally, tumors are not often palpable thus do not provide sensory feedback during percutaneous or surgical treatment. Thus, additional percutaneous procedure in radiology is performed for preoperative localization (such as wire localization, radioactive seed localization, MagSeed [EndoMag], Savi Scout [Merit medical], Smart Clip [elucent]).

The system can allow the operator to visualize respiratory compensation integration and live updates to deformation via photogrammetry, videogrammetry, ultrasound, MRI, and/or use of seeded targets generated by Finite Element Analysis or similar deformation algorithms. The practioner can also see how the tissue changes when application of an ultrasound probe, supine vs prone imaging changes, or any other application of force via FEA or similar means. In breast applications, it is important to display the predicted lumpectomy or ablation change for plastic surgery reasons. The system can provide heat maps of tissue deformation relative to target and the effect of the medical device displayed in AR with data metrics, live in AR tracking of multi-target or multi-probe applications, and haptic feedback relative to the registered target. The heat maps can be accompanied with descriptive analytics. The power of this is to overlay different time points within a procedure (or animate as the procedure takes place), compare similar procedures, and compare novice to expert within or across users/patients, as non-limiting examples.

The spatial analytics of the system can be utilized in dental and facial reconstruction. One could sync up any number of sensors (i.e., force transducers on a hand or robotic tool) when performing dental or orthopedic implants at the location in 3D space, record them in a heat map form (point cloud of data changing over time), and analyze them in a simple histogram representation. The system can also utilize spatial sound relative to dental applications. The system can identify and cancel unwanted noise (e.g., medical machines, motors, bone saws, or any other sound/frequency that would cause undo cognitive load or distraction especially when doing therapy delivery.) The operator can interact with the visualization to see how a stressor, implant device, or implant could change flow intra-and post-procedural. The system can also show where the sound is coming from graphically and intensity level.

Spatial analytics and sound analytics of the system can be very useful in veterinary medicine due to the within and across variation of animal species. There are not often highly skilled surgeons/interventionalists in facilities or on-site, for example on farms or the field making the present technology particularly advantageous. The digital data rich holographic twins of the system can be displayed and interacted with an external expert.

### Application 2: Radiomic Signatures Used ss Texture Maps on Segmented Tumors

Radiomic signatures of heterogeneous tumors can be used to characterize subregions referred to as habitats. Image processing methods, including semantic and agnostics feature detection, can be applied to local pixel “neighborhoods” of a segmented tumor volume to characterize habitats with feature vectors. Radiomics signatures can be visualized during image guided procedures for potentially improved targeting of biopsy and treatment.

The system can segment a 3D volume of pre-procedurally imaged tumors. 3D image processing techniques can extract traits or feature vectors based on texture, shape, intensity, and wavelets to form a radiomics signature. A texture map for the tumor surface can be constructed based on relevant Radiomic signature. The operator can select and visualize radiomic signatures that can be predictive of, e.g., tumor aggressiveness. The texture map can be applied to the tumor material to highlight where the tumor should be sampled or treated.

Identification of a particular type or portion of a tumor can incorporate a classification learning algorithm that takes a collection of labeled examples as inputs and produces a model that can take an unlabeled example as input and either directly output a label or output a number that can be used by the analyst to deduce the label.

The system can project or display the radiomic metrics (feature descriptors from annotations) that correlate texture to specific feature (shape, entropy, etc.). The projections can be utilized in pre-procedure planning, intra-procedure decision making, and post-procedure analysis. The system can be continually updated, through the feedback loop, to continue improving the radiomic projections based on the post-procedure analysis. The system can utilize information of which features are most relevant to biopsy or treat and use this to texture the sub-surfaces of the heterogenous target structure.

In veterinary medicine there is a lack of the same quantity, quality, and applicable data. In these cases, the system can incorporate transfer learning to the radiomic models. In transfer learning, an existing model trained on some dataset is selected and then adapted to a desired model to predict examples from another dataset, different from the one the model was built on. For example, a first model can be trained to recognize (and label) tumors of a human on a big, labeled data set. A smaller data set can be compiled for the second model (tumors of animals). Machine learning can then be utilized to modify the first model with attributes of the second model, to make the second model workable.

### Application 3: Human in the Loop Machine Learning During Shared Experiences

Shared experience on the system can enable collaborative mentors to provide advice on surgical methods and maneuvers. A machine learning (ML) model can also be used to assist with decisions, image interpretation, or classification, however, ground truth data can be sparce, uncertain, or unknown. The system can be used in a Human-In-the-Loop method to improve the accuracy of data input to the model and improve its training.

A mentee can use a model and the mentor can observe the same image or 3D holographic guidance and navigation scene during a procedure. Interpretation is provided by the model and the mentor, for example. The interpretation by the mentor can used to compute a loss function, DICE score, and optimize the model. The mentor, an expert in the task under consideration, can subsequently select test samples with the highest decision confidence for re-training the model. The shared experience can include more than one mentor. For example, the surgical maneuvers suggested by the mentor are recorded and used to provide feedback to a machine learning algorithm.

Some classification algorithms (like decision tree learning, logistic regression, or SVM) build the model using the whole dataset at once. If one has additional labeled examples, there may be the need to rebuild the model from scratch. Other algorithms (such as Naïve Bayes, multilayer perceptron, SGDClassifier/SGDRegressor, Passive AggressiveClassifier/PassiveAggressiveRegressor in scikit-learn) can be trained iteratively, one batch at a time. Once new training examples are available, one can update the model using only the new data.

k-Nearest Neighbors (kNN) is a non-parametric learning algorithm. Contrary to other learning algorithms that allow discarding the training data after the model is built, kNN keeps all training examples in memory. Other popular distance metrics include Chebychev distance, Mahalanobis distance, and Hamming distance.

Sometimes there are only examples of one class, and one would like to train a model that would distinguish examples of this class from everything else. One-class classification, also known as unary classification or class modeling, tries to identify objects of a specific class among all objects, by learning from a training set containing only the objects of that class. One-class classification learning algorithms are used for outlier detection, anomaly detection, and novelty detection. There are several one-class learning algorithms. The most widely used in practice are one-class Gaussian, one-class k-means, one-class kNN, and one-class SVM.

Active learning is usually applied when obtaining labeled examples is costly. That is often the case in the medical or financial domains, where the opinion of an expert may be required to annotate patients' or customers' data. The idea is to start learning with few labeled examples, and a large number of unlabeled ones, and then label only those examples that contribute the most to the model quality.

When the labeled/annotated data is obtained, it can be divided into distinct sets: 1) training set, 2) validation set, and 3) test set. Initially, one can shuffle the examples and split the dataset into three subsets: training, validation, and test. The training set is usually the biggest one, which can be used to build the model. The validation and test sets are roughly the same sizes, much smaller than the size of the training set. The learning algorithm cannot use examples from these two subsets to build the model.

The following method could be used in this process since sufficient data may be limited given expert input limitations. In semi-supervised learning (SSL), a small fraction of the dataset is labeled and most of the remaining examples are unlabeled. The goal is to leverage a large number of unlabeled examples to improve the model performance without asking for additional labeled examples. neural network architecture that attained such a remarkable performance is called a ladder network. To understand ladder networks, one has to understand what an autoencoder is. An autoencoder is a feed-forward neural network with an encoder-decoder architecture, an example of this would be a Generative Adversarial Network (GAN). It is trained to reconstruct its input.

For example, when giving input to a physician via similar examples from themselves or experts, “Learning to Recommend” is an approach to building recommender systems. Usually, there is a user who consumes content. A user's history of consumption is provided, and new content is suggested to the user that they would like. Traditionally, two approaches were used to give recommendations: content-based filtering and collaborative filtering. Content-based filtering consists of learning what users like/need based on the description of the content they consume. The content-based approach has many limitations. For example, the user can be trapped in the so-called filter bubble: the system will always suggest to that user the information that looks very similar to what user already consumed.

Collaborative filtering has a significant advantage over content-based filtering: the recommendations to one user are computed based on what other users consume or rate. For instance, if two users gave high ratings to the same ten procedures, then it's more likely that user I will appreciate new procedures recommended based on the tastes of the user 2 and vice versa. The drawback of this approach is that the content of the recommended items is ignored. In collaborative filtering, the information on user preferences is organized in a matrix. Each row corresponds to a user, and each column corresponds to a piece of content that user rated or consumed. Usually, this matrix is huge and extremely sparse, which means that most of its cells aren't filled (or filled with a zero). The reason for such a sparsity is that most users consume or rate just a tiny fraction of available content items. It is very hard to make meaningful recommendations based on such sparse data. Most real-world recommender systems use a hybrid approach: they combine recommendations obtained by the content-based and collaborative filtering models.

Two effective recommender system learning algorithms are factorization machines (FM) and denoising autoencoders (DAE). A denoising autoencoder can include a neural network that reconstructs its input from the bottleneck layer. The fact that the input is corrupted by noise while the output should not be makes denoising autoencoders an ideal tool to build a recommender model.

### Application 4: Characterization and Reproduction of Respiratory Phase in a CT Data Set

During an interventional procedure, the patient is undergoing respiratory motion, but the CT data set used for guidance and navigation is typically acquired at a particular breath-hold. It can be desirable to reproduce the breath-hold or ventilation pause so that the CT-based holograms used for guidance and navigation have an increased congruence with the physical anatomy.

A deep learning model is trained to estimate the respiratory phase that occurred during acquisition of a CT data sets. The training set includes CT data sets that were acquired with at a known breath-hold. Specifically, the training CT data sets that were acquired with respiratory phases ranging from deep inhalation to deep exhalation, with output classification from −1 to 1 respectively, in increments of 0.1 amplitude units (AU).

For an individual patient, a CT data set is input to the model to determine the respiratory phase in AU. During the procedure, a motion sensor (including accelerometers or non-contact sensors like depth and IR cameras like HoloLight attachment on HoloLens 2 or separate Kinect camera) or air bellows or electromagnetic sensors are placed on the chest surface. An indication is provided to the practitioner when the patient respiratory phase as measured by the motion sensor is matched with phase that was estimated by the ML model.

In addition to regression models mentioned above (decision tree learning, SVM, kNN, etc.), other more suitable solutions for velocity changes or acceleration changes could be model with sigmoid functions via polynomial regression or an AI method such as logistic regression (a.k.a., sigmoid function).

### Application 5: Real-Time Intra-Procedural Access to Patient Data and Actionable Cohort Data Insights

The system can utilize patient data, which can be associated with an individual patient and/or from a defined cohort of patients to make pre-procedural and intra-procedure decisions. The system can import and select patient data from an Electronic Health Record (EHR). The system can utilize actionable data insights from Fast Healthcare Interoperability Resources® (FHIR) based on population data of similar patients, procedures, and/or diagnoses shared across a cohort of multiple hospital systems to support real-time clinical decision making. This information can be augmented to a virtual display near the patient during the procedure.

### Application 6: Ablation Zone Planning

Practitioners can utilize the system to plan ablation zones. Practitioners can use holographic anatomy populated from CT scan, tracked instruments to set preliminary ablation zone. The ablation zone can then be stamped onto the ultrasound system. The ablation zone can bidirectionally updated on both ultrasound and holographic anatomy. Ablation zone data can be saved on the US system or other hospital system and specific user preferences are created/updated. For future procedures, user preferences can be used to populate ablation trajectory. Preferences include avoiding critical structures, gradients/grow rate, input parameters, etc. The continuous data loop would allow providers to be able to use and apply previous ablation zone practices to guide current procedures.

### Additional Examples

It should also be appreciated that the system and feedback loop of the present technology can be described in the context of various additional examples as described further herein, and also provided with reference to the figures enclosed herewith.

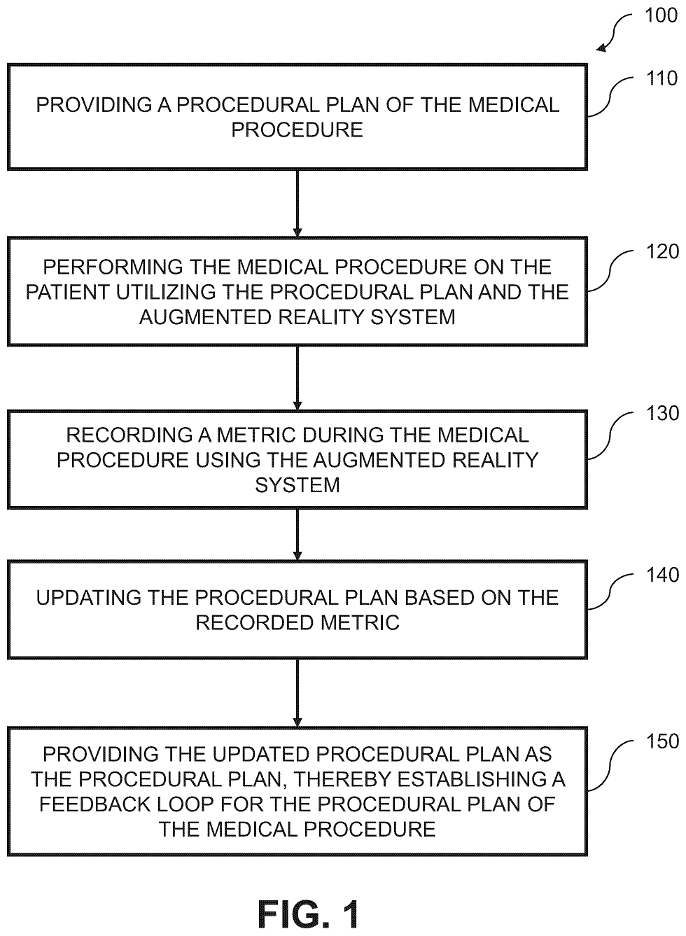

FIG. 1 is a flowchart of an example method 100 for enhancing surgical precision and outcomes in a medical procedure using an augmented reality system. At step 110, the method 100 includes providing a procedural plan of the medical procedure. The procedural plan can include a pre-operative plan of the medical procedure. The procedural plan can also include a general plan of the procedure based on the knowledge of the practitioner. At step 120, the method 100 includes performing the medical procedure on the patient utilizing the procedural plan and the augmented reality system. At step 130, the method 100 includes recording a metric during the medical procedure using the augmented reality system. At step 140, the method 100 includes [updating the procedural plan based on the recorded metric. At step 150, the method 100 includes providing the updated procedural plan as the procedural plan, thereby establishing a feedback loop for the procedural plan of the medical procedure.

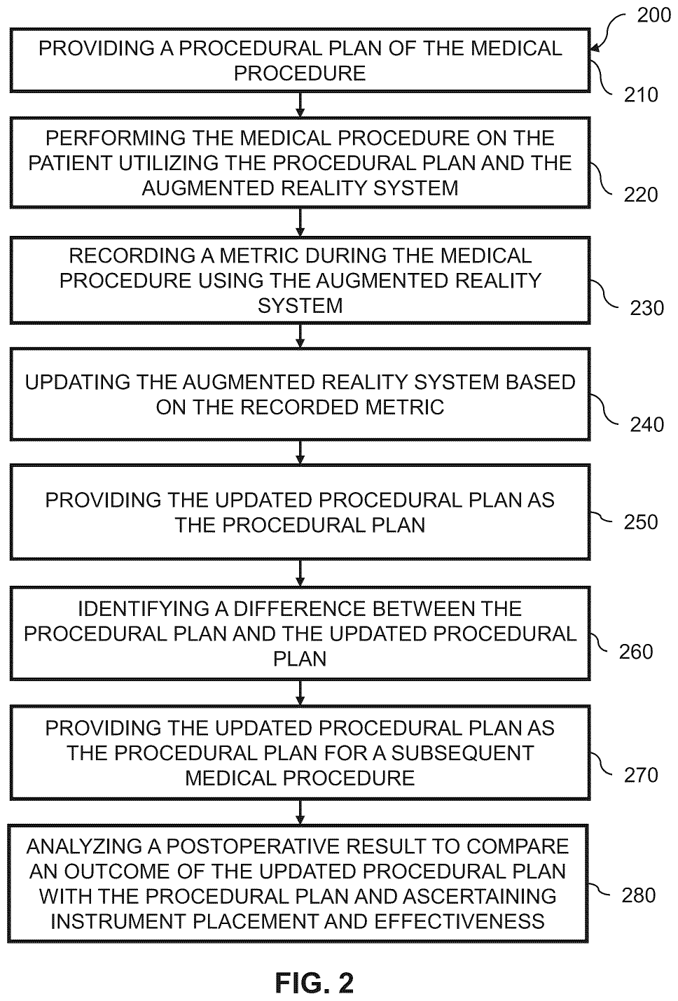

FIG. 2 is a flowchart of an example method 200 for performing and updating a medical procedure on a patient by a practitioner using an augmented reality system.

At step 210, the method 200 includes providing a procedural plan of the medical procedure]

At step 220, the method 200 includes performing the medical procedure on the patient utilizing the procedural plan and the augmented reality system. The augmented reality system can provide intraoperative guidance during the medical procedure by providing an augmented reality display of an aspect of the procedural plan and tracking of a surgical instrument. This step can further include alerting the practitioner to a deviation from the procedural plan through visual or auditory cues, wherein the augmented reality system includes a head-mounted display for rendering a holographic representation to the practitioner performing the medical procedure, and the head-mounted display is configured to overlay the holographic representation within the practitioner's view of the patient receiving the medical procedure.

At step 230, the method 200 includes recording a metric during the medical procedure using the augmented reality system, wherein the metric recorded includes data selected from the group consisting of instrument positioning metrics, instrument interaction metrics, operative action metrics, outcome-related metrics, procedure efficiency metrics, and system interaction metrics.

At step 240, the method 200 includes updating the procedural plan based on the recorded metric. At step 250, the method 200 includes providing the updated procedural plan as the procedural plan, thereby establishing a feedback loop for the procedural plan of the medical procedure. At step 260, the method 200 includes identifying a difference between the procedural plan and the updated procedural plan. At step 270, the method 200 includes providing the updated procedural plan as the procedural plan for a subsequent medical procedure, including adjusting a holographic representation by the augmented reality system, and the holographic representation includes a three-dimensional model of a portion of an anatomy of the patient derived from medical imaging data. At step 280, the method 200 includes analyzing a postoperative result to compare an outcome of the updated procedural plan with the procedural plan and ascertaining instrument placement and effectiveness of the instrument, wherein the augmented reality system is further configured to provide a postoperative review interface to display the recorded metric relative to the procedural plan and provide adjustment of the procedural plan.

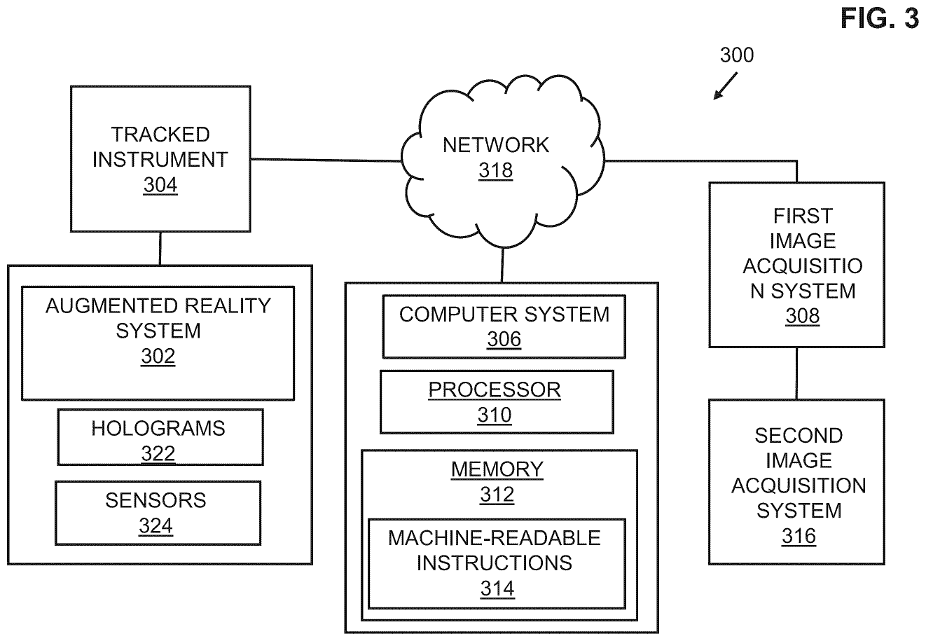

As shown in FIG. 3, in a first example a system 300 for holographic augmented reality visualization and guidance includes an augmented reality system 302, a tracked instrument 304, a computer system 306, and a first image acquisition system 308. In certain embodiments, the system 300 can further include a second image acquisition system 316. Each of the augmented reality system 302, the tracked instrument 304, the first image acquisition system 308, and the second image acquisition system 316 can be selectively or permanently in communication with the computer system 306, for example, via a computer network 318. Other suitable instruments, tools, equipment, sub-systems, and the like for use with the holographic augmented reality visualization and guidance system 300, as well as other network means including wired and wireless means of communication between the components of the holographic augmented reality visualization and guidance system 300, can also be employed by the skilled artisan, as desired. The tracked instrument 304 is an interventional device that is sensorized so that that both a location and an orientation of the tracked instrument 304 can be determined by the computer system 306. The computer system 306 can be utilized to record the metrics during the procedure and can be used to apply the updates to the procedural plan.

With continued reference to FIG. 3, the computer system 306 of the first example can include a memory 312 on which tangible, non-transitory, machine-readable instructions 314 can be stored. The memory 312 can include one or more types of memory and can include any type suitable to the local application environment. Examples include where the memory 312 can include various implementations of volatile and/or nonvolatile data storage technology, such as a semiconductor-based memory device, a magnetic memory device and system, an optical memory device and system, fixed memory, and removable memory. For example, the memory 312 can include one or more of random access memory (RAM), read only memory (ROM), static storage such as a magnetic or optical disk, hard disk drive (HDD), or any other type of non-transitory machine or computer readable media, as well as combinations of the aforementioned types of memory. Instructions stored in the memory 312 can include program instructions or computer program code that, when executed by the processor 310, enables the system 300 for holographic augmented reality visualization and guidance to perform tasks as described herein.

The machine-readable instructions 314 can include one or more various modules. Such modules can be implemented as one or more of functional logic, hardware logic, electronic circuitry, software modules, and the like. The modules can include one or more of an augmented reality system module, an image acquiring module, an instrument tracking module, an image dataset registering module, a hologram rendering module, an image registering module, a trajectory hologram rendering module, and/or other suitable modules, as desired.

The computer system 306 can be in communication with the augmented reality system 302, the tracked instrument 304, and the first image acquisition system 308, and the second image acquisition system 316, for example, via the network 320, and can be configured by the machine-readable instructions 314 to operate in accordance with various methods for holographic augmented reality visualization and guidance in performing a medical procedure on an anatomical site of a patient by a user as described further herein. The computer system 306 can be separately provided and spaced apart from the augmented reality system 302, or the computer system 306 can be provided together with the augmented reality system 302 as a singular one-piece unit or integrated with other systems, as desired.

With continued reference to FIG. 3, the augmented reality system 302 can be configured to render a plurality of holograms 322 in operation of the system 300 in accordance with the first example of the present disclosure. In particular, the augmented reality system 302 can include a mixed reality (MR) display such as one or more MR smart glasses or MR head-mounted displays. Further nonlimiting examples of the augmented reality system 302 can include the Magic Leap One® or versions of the Microsoft HoloLens®. It should be appreciated that other types of MR displays can be used for the augmented reality system 302, as long as they are capable of superimposing computer-generated imagery, including holograms, over real-world objects. Additionally, although the augmented reality system 302 can be described primarily as including a head-mounted display, it should be understood that other types of displays that are not head-mounted, but which are capable of generating and superimposing holograms 322 over real-world views, can also be employed, as desired.

It should be appreciated that in certain embodiments the augmented reality system 302 can also include one or more positional sensors 324. One or more positional sensors 324 of the augmented reality system 302 can be configured to determine various positional information for the augmented reality system 302, such as the approximated position in three-dimensional (3D) space, the orientation, angular velocity, and acceleration of the augmented reality system 302.

As shown in FIG. 3, the computer system 306 according to the first example can be in communication with the augmented reality system 302 and the tracked instrument 304. The computer system 306 can be configured to store and generate the operating information, either through manual intervention by the user and/or other medical professionals or automatically based on machine-readable instructions 314 encoded within the memory 312. For example, the operating information can be generated in the augmented reality system 302 depending on a sensor-determined position and/or orientation of the tracked instrument 304, such as by using algorithms, artificial intelligence (AI) protocols, or other user-inputted data or thresholds.

In a second example, as also depicted in FIG. 3, the augmented reality system 300 is designed to enhance precision and outcomes in medical procedures. This system comprises a head-mounted device of the augmented reality system 302 equipped with a display for rendering holographic images. Integral to the head-mounted device of the augmented reality system 302 are the processor 310 and the non-transitory memory 312, which facilitate the operation and data management of the system.

The tracked instrument 304 may be part of a tracking system, integrated with the head-mounted device of the augmented reality system 302, which is configured to provide real-time tracking data of a medical instrument in relation to the anatomy of a patient. This enables precise navigation and positioning of surgical instruments during procedures. The medical instrument can include other treatment therapies including placement of implant, therapy, markers, etc.

With continued reference to the second example, the machine-readable instructions 314, stored within the non-transitory memory 312 and executed by the processor 310, enable the head-mounted device of the augmented reality system 302 to perform several critical functions. These functions include transforming tracking data from the tracked instrument 304 of the tracking system into a coordinate system used by the head-mounted device of the augmented reality system 302, accessing and transforming pre-operative image data of the patient's anatomy into the coordinate system of the head-mounted device of the augmented reality system 302, and generating a three-dimensional holographic representation of the patient's anatomy and the medical instrument.

The augmented reality system 300 overlays this three-dimensional holographic representation onto the patient's anatomy within the field of view of a practitioner, providing intraoperative guidance. The system 300 is also capable of recording intraoperative metrics during the medical procedure, which include data such as instrument positioning metrics, instrument interaction metrics, operative action metrics, outcome-related metrics, procedure efficiency metrics, and system interaction metrics.

Also according to the second example, as may be illustrated by FIG. 3, these recorded metrics are utilized to update the procedural plan, refining surgical approaches and instrument paths. This updated procedural plan is then stored, establishing a feedback loop for continuous improvement in surgical practices.

Additionally, the augmented reality system 300 includes a communication interface for the reception of pre-operative image data and the transmission of recorded intraoperative metrics. A user interface is also provided, allowing the practitioner to interact with the augmented reality system 300 to adjust the procedural plan and view the three-dimensional holographic representation during the medical procedure.

Advantageously, the augmented reality system 300 of both the first example and the second example, as illustrated in FIG. 3, offers a comprehensive solution for improving the accuracy and efficacy of medical procedures through advanced visualization, real-time tracking, and iterative learning from recorded surgical metrics.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods may be made within the scope of the present technology, with substantially similar results.

What is claimed is:

1. A method of performing and updating a medical procedure on a patient by a practitioner using an augmented reality system, the method comprising:
providing a procedural plan of the medical procedure;
performing the medical procedure on the patient utilizing the procedural plan and the augmented reality system;
recording a metric during the medical procedure using the augmented reality system to provide a recorded metric;
updating the procedural plan based on the recorded metric to provide an updated procedural plan; and
using the updated procedural plan as the procedural plan, thereby establishing a feedback loop for the procedural plan of the medical procedure.

2. The method of claim 1, further comprising:
identifying a difference between the procedural plan and the updated procedural plan.

3. The method of claim 1, further comprising a step of:
providing the updated procedural plan as the procedural plan for a subsequent medical procedure.

4. The method of claim 1, wherein the augmented reality system provides intraoperative guidance during the medical procedure by providing an augmented reality display of an aspect of the procedural plan and tracking of a medical instrument.

5. The method of claim 4, wherein the intraoperative guidance includes a step of alerting the practitioner to a deviation from the procedural plan.

6. The method of claim 5, wherein the step of alerting includes visual or auditory cues.

7. The method of claim 4, wherein:
providing the updated procedural plan as the procedural plan includes a step of adjusting a holographic representation by the augmented reality system; and
the method further includes a step of providing the updated procedural plan as the procedural plan for a subsequent medical procedure.

8. The method of claim 7, wherein the holographic representation includes a three-dimensional model of a portion of an anatomy of the patient derived from medical imaging data.

9. The method of claim 1, wherein the recorded metric obtained during the medical procedure includes data selected from the group consisting of instrument positioning metrics, instrument interaction metrics, operative action metrics, outcome-related metrics, procedure efficiency metrics, and system interaction metrics.

10. The method of claim 1, wherein providing the updated procedural plan as the procedural plan includes a step of adjusting a holographic representation by the augmented reality system based on the recorded metric.

11. The method of claim 1, wherein the augmented reality system includes a head-mounted display for rendering a holographic representation to the practitioner performing the medical procedure.

12. The method of claim 11, wherein the head-mounted display is configured to overlay the holographic representation within a view by the practitioner of the patient receiving the medical procedure.

13. The method of claim 1, wherein providing the updated procedural plan as the procedural plan, thereby establishing the feedback loop for the procedural plan of the medical procedure, includes a step of analyzing a postoperative result to compare an outcome of the updated procedural plan with the procedural plan within the step of providing the procedural plan and, wherein the step of analyzing the postoperative result further includes steps of ascertaining a placement of a medical instrument and ascertaining an effectiveness of the medical instrument.

14. The method of claim 1, wherein the augmented reality system is further configured to track a position and orientation of a medical instrument in real-time during the medical procedure.

15. The method of claim 1, wherein the augmented reality system is further configured to integrate and display data from an imaging system and a tracking system.

16. The method of claim 1, wherein the augmented reality system is further configured to provide a postoperative review interface to display the recorded metric relative to the procedural plan and provide adjustment of the procedural plan.

17. The method of claim 1, wherein the augmented reality system utilizes a machine learning algorithm to modify a holographic representation and intraoperative guidance of the procedural plan based on a plurality of iterations of:
recording the metric during the medical procedure using the augmented reality system;
updating the procedural plan based on the recorded metric; and
providing the updated procedural plan as the procedural plan, thereby establishing the feedback loop for the procedural plan of the medical procedure.

18. The method of claim 1, wherein the augmented reality system is further configured to provide a preemptive action relative to the procedural plan using predictive analytics based on the step of providing the updated procedural plan as the procedural plan, thereby establishing the feedback loop for the procedural plan of the medical procedure.

19. A method of performing and updating a medical procedure on a patient by a practitioner using an augmented reality system, the method comprising:
providing a procedural plan of the medical procedure;
performing the medical procedure on the patient utilizing the procedural plan and the augmented reality system, wherein the augmented reality system provides intraoperative guidance during the medical procedure by providing an augmented reality display of an aspect of the procedural plan and tracking of a medical instrument, including alerting the practitioner to a deviation from the procedural plan through visual or auditory cues, wherein the augmented reality system includes a head-mounted display for rendering a holographic representation to the practitioner performing the medical procedure, and the head-mounted display is configured to overlay the holographic representation within a view by the practitioner of the patient receiving the medical procedure;
recording a metric during the medical procedure using the augmented reality system to provide a recorded metric, wherein the recorded metric includes data selected from the group consisting of instrument positioning metrics, instrument interaction metrics, operative action metrics, outcome-related metrics, procedure efficiency metrics, and system interaction metrics;
updating the procedural plan based on the recorded metric to provide an updated procedural plan;
providing the updated procedural plan as the procedural plan, thereby establishing a feedback loop for the procedural plan of the medical procedure;
identifying a difference between the procedural plan and the updated procedural plan;
providing the updated procedural plan as the procedural plan for a subsequent medical procedure, including adjusting a holographic representation by the augmented reality system, and the holographic representation includes a three-dimensional model of a portion of an anatomy of the patient derived from medical imaging data;
analyzing a postoperative result to compare an outcome of the updated procedural plan with the procedural plan and ascertaining placement of the medical instrument and effectiveness of the medical instrument, wherein the augmented reality system is further configured to provide a postoperative review interface to display the recorded metric relative to the procedural plan and provide adjustment of the procedural plan.

20. An augmented reality system for enhancing precision and outcomes in medical procedures, comprising:
a head-mounted device equipped with a display for rendering holographic images, the head-mounted device including a processor and a non-transitory memory;
a tracking system integrated with the head-mounted device, the tracking system configured to provide real-time tracking data of a medical instrument in relation to an anatomy of a patient;
machine-readable instructions stored in the non-transitory memory that, when executed by the processor, enable the head-mounted device to
transform tracking data from the tracking system into a coordinate system used by the head-mounted device,
access and transform pre-operative image data of the anatomy of the patient into the coordinate system of the head-mounted device,
generate a three-dimensional holographic representation of the anatomy of the patient and the medical instrument based on the transformed tracking data and pre-operative image data,
overlay the three-dimensional holographic representation onto the anatomy of the patient within a field of view of a practitioner to provide intraoperative guidance,
record intraoperative metrics during the medical procedure to provide a recorded metric,
update a procedural plan based on the recorded metric to provide an updated procedural plan to refine surgical approaches and instrument paths, and
establish a feedback loop by storing the updated procedural plan for use in subsequent medical procedures, thereby creating a cycle of continuous improvement in surgical practices;
a communication interface for receiving the pre-operative image data and transmitting the recorded intraoperative metrics; and
a user interface allowing the practitioner to interact with the augmented reality system to adjust the procedural plan and view the three-dimensional holographic representation during the medical procedure.
